(19) United States
(12) Patent Application Publication
(10) Pub. No.: US 2004/0133186 A1
(43) Pub. Date: Jul. 8, 2004

(54) Programmed pulsed infusion methods and devices

(76) Inventor: Shailendra Joshi, Edgewater, NJ (US)

Correspondence Address: Leslie Gladstone Restaino, 163 Madison Avenue, P.O. Box 1989, Morristown, NJ, 07962-1989, US

(21) Appl. No.: 10/337,677

(22) Filed: Jan. 6, 2003

(51) Int. Cl. A61K 9/22

(52) U.S. Cl. 604/890.1; 604/500; 705/2

(57) ABSTRACT

This invention disclosed herein provides methods, devices, software products, and systems for infusing a fluid into a blood vessel is provided that includes the step of administering the fluid into the blood vessel in programmed pulses. The programmed pulses generally defined by programmed pulse variables that include a fluid flow rate, a frequency, and a duration. Values of the programmed pulse variables may be determined based at least in part on a fluid property of the fluid to be infused that is relevant to streaming, blood flow in the blood vessel to be infused, a catheter size, or a patient profile.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyrights whatsoever.

### BACKGROUND OF THE INVENTION

The invention generally relates to infusion techniques. Specifically, the invention discussed herein relates to methods and devices that generate more predictable drug concentrations downstream by introducing or administering a drug dissolved in fluid into a blood vessel in a manner to compensate for or otherwise overcome streaming and variable velocity related to tissue uptake.

In certain blood vessels, such as the carotid artery, the velocity or flow of the blood therein is such that fluid introduced into the blood vessel, particularly in small quantities, has a tendency to follow the stream of the blood flow and pass a site targeted for the fluid. Streaming is exhibited, particularly in laminar flow fluid patterns due to the variable velocity pattern occurring with a flowing fluid. Streaming may be explained with reference to FIG. 1, which shows a section of a blood vessel 102 with a laminar fluid flow along the X-axis. The velocity of the fluid varies as shown by the fluid velocity (V) profile 104, which is a function of the distance (D) along the Y-axis. A small quantity of fluid 108 introduced into the flow has a tendency to follow the direction of the higher velocity streamlines 104 and pass a possible target 112 for the fluid, rather than in the direction of the lower velocity streamline 110 to the target 112. Because of streaming, delivering fluids, such as drugs, in small quantities, particularly via the carotid artery, has proven to be unreliable and the effects of the drugs consequently not consistent.

One attempt to overcome streaming has been to infuse drugs by periodically pulsing the fluid at high infusion rates and phased with diastole, in an attempt to introduce the fluid into the carotid artery at a sufficient rate to overcome the streaming in at the time the blood flow at the injection site is at the lowest. This method of overcoming streaming, however, has numerous shortcomings. Although low average infusion rates are achieved, the fluid must still be infused at a high rate during the pulse, which with respect to anesthetic drugs may have significant cardiovascular effects. Additionally, diastole phased infusion requires complex equipment to sense and phase the high-infusion-rate pulses in synch with diastole, which may vary during infusion. There is therefore a need for infusion methods and devices to overcome streaming which deliver fluid at lower infusion rates and/or independent of diastole.

Moreover, during traditional intra-aeterial infusions, the uptake of the drug by the tissue is determined by the biological properties of the drug. For example, in the brain there are specific transport mechanisms that buffer high concentrations of drugs injected into the carotid artery, such that tissue concentrations of the drug are not increased. The method of drug infusion described herein utilizes the physical and biological properties of the drug to define the characteristics of the pulse.

### SUMMARY OF THE INVENTION

This invention overcomes the shortcomings in the art by generally providing methods, devices, and software products for use in infusion therapy in a manner less prone to streaming, provides greater efficient use of the fluid, and with respect to drug infusion, in a manner providing greater efficient uptake to a targeted organ, such as the brain, and less systemic side affects.

In one aspect of the invention, a method of infusing a fluid into a blood vessel is provided that includes the step of administering the fluid into the blood vessel in programmed pulses. The programmed pulse characteristics may be defined by programmed pulse variables that include a flow rate for the drug-containing fluid, a frequency, and a duration. Values of the programmed pulse variables may be determined based at least in part on a fluid property of the fluid to be infused that is relevant to streaming, blood flow in the blood vessel to be infused, a catheter size, or a patient profile. In one embodiment, the fluid is administered in programmed pulses independent of diastole.

The methods described herein may be used in a variety of blood vessels. The methods are particularly useful in treating conditions associated with a patient's head, such stroke after thrombosis, cancer, cerebral vasospasm, infection, and localization of brain function. In these instances, the fluid may be delivered in programmed pulses into a patient's carotid artery. In one embodiment of the invention, the drug is administered at a fluid flow rate of about 2% to about 5% of arterial blood flow of the carotid artery. In one embodiment, the fluid is administered in programmed pulses at a duration of about one to five seconds.

Treatment generally relates to the discovery and application of remedies to manage or care for an injury or disease. The invention as described herein may be used to produce clinical imaging using X-rays, ultrasound, computed tomography, magnetic resonance, radionuclide scanning, thermography, etc., in connection with surgical procedures, such as to administer an anesthetic, or in connection with non-invasive treatments, such as to administer chemotherapy to tread cancer. It is reasonably understood that those skilled in the art may apply the present invention in a variety of treatments. The examples provided herein are therefore merely for illustration and not to be viewed as limitations.

In one embodiment, the fluid property of the fluid to be infused that is relevant to streaming includes a static fluid property or a kinetic fluid property. Alternatively, or in addition, where the fluid is a drug, the fluid property of the fluid to be infused that is relevant to streaming includes a pharmacological property of the drug. Additionally, where the fluid is a drug, the fluid may be administered in programmed pulses at the duration based on a transfer rate of the drug across a blood brain barrier or at the frequency based on a rate of elimination of the drug from a brain. In one embodiment, where the fluid is a drug, the concentration of the drug is determined based on a kinetic property of the drug and/or based on a therapeutic index of the drug.

In one aspect of this invention, a device for administering a fluid in programmed pulses, i.e., a programmed pulse infusion device, is provided. The programmed pulse infusion device includes a pump, a controller with associated memory interfacing with the pump, and a fluid reservoir containing a fluid feeding the pump. The controller provides a drive signal for the pump to deliver the fluid to be infused into a blood vessel in programmed pulses that are defined by programmed pulse variables, which include a fluid flow rate, a frequency, and a duration. The values of the programmed pulse variables may be determined based at least in part on a fluid property of the fluid to be infused relevant to streaming, blood flow in the blood vessel to be infused, a catheter size, or a patient profile. The programmed pulse infusion device may deliver the fluid in programmed pulses independent of diastole and for infusion into a carotid artery.

In one embodiment, the values of the programmed pulse variables base on the fluid property of the fluid to be infused relevant to streaming includes a static fluid property and/or a kinetic fluid property. Where the fluid is a drug, the fluid property of the fluid to be infused relevant to streaming may be a pharmacological property of the drug. Additionally, where the fluid is a drug, the duration may be based on a transfer rate of the drug across a blood brain barrier and the frequency may be based on a rate of elimination of the drug from a brain.

In one embodiment, the programmed pulse infusion device includes a control solenoid that interfaces with the controller. The controller may provide an actuating signal to the solenoid to deliver the fluid in programmed pulses. In this instance, the pump may be a pneumatic actuated pump. The programmed pulse infusion device may also include a bubble trap, which may be used for removing air bubbles from the fluid being delivered and may include a pressure sensor interfacing with the controller for observing the fluid delivery pressure. In one embodiment, the fluid reservoir is a syringe and the pneumatic actuated pump includes a plunger that is extended by an actuating force toward the syringe to deliver the fluid from the syringe in programmed pulses.

In one aspect of this invention, a software product or program code is provided on a computer readable medium that when executed enables a user to determine values of programmed pulse variables for infusing the fluid in a blood vessel in programmed pulses that are defined by the programmed pulse variables that include a fluid flow rate, a frequency, and a duration. The values of the programmed pulse variables may be determined at least in part on a fluid property of the fluid to be infused that is relevant to streaming, blood flow in the blood vessel to be infused, a catheter size, or a patient profile. The values of the programmed pulse variables may be determined independent of diastole. In one embodiment, the program code enables users to determine values of the programmed pulse variables for infusing the fluid into a carotid artery.

In one embodiment, the fluid property of the fluid to be infused that is relevant to streaming includes a static fluid property or a kinetic fluid property, and where the fluid is a drug, the fluid property of the fluid to be infused relevant to streaming may include a pharmacological property of the drug. Additionally, where the fluid is a drug, the drug may be infused in programmed pulses in a carotid artery to treat stroke after thrombosis, cancer, cerebral vasospasm, infection, or localization of brain function.

In one aspect of this invention, a computer system is provided that includes a controller and associated computer memory, and a computer readable medium that is accessible to the controller. Stored on the computer readable medium may be a database or databases including data of fluid properties for a fluid that may be infused into a blood vessel relevant to streaming, and values of programmed pulse variables based on a patient profile, a blood flow rate in the blood vessel, a fluid property for the fluid relevant to streaming, and/or a catheter size. The database may be accessed or is accessible to enable users to determine the values of programmed pulse variables for infusing the fluid in the blood vessel in programmed pulses that are defined by the programmed pulse variables that include a fluid flow rate, a frequency, and a duration.

In one embodiment, the values of programmed pulse variables may be based on a fluid property for the fluid relevant to streaming that may include a static fluid property or a kinetic fluid property. Where the fluid is a drug, the values of programmed pulse variables may be based on a fluid property for the fluid relevant to streaming that includes a pharmacological property of the drug. Additionally, where the fluid is a drug, the duration of a programmed pulse may be based on a transfer rate of the drug across a blood brain barrier and the frequency of a programmed pulse may be based on a rate of elimination of the drug from the brain.

### BRIEF DESCRIPTION OF THE FIGURES

The invention is illustrated in the figures of the accompanying drawings, which are meant to be exemplary, and not limiting, in which like references refer to like or corresponding parts, and in which:

FIG. 1 is a sectional view of a blood vessel showing the streaming effect on a fluid in the blood vessel;

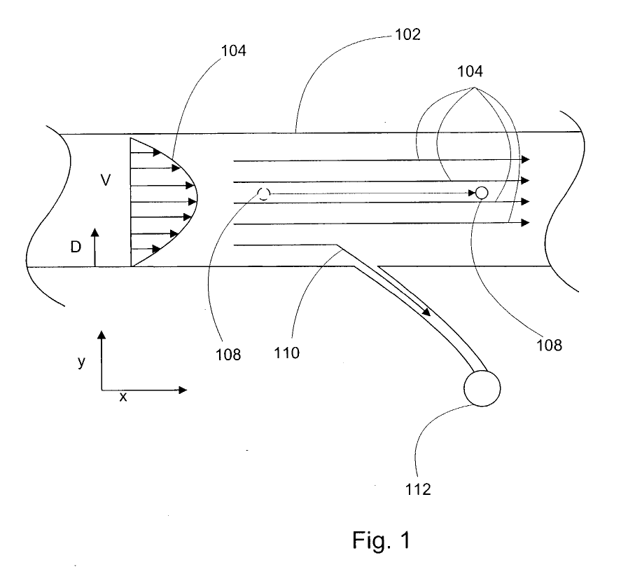

FIG. 2 is a flowchart of a method for administering a fluid in programmed pulses, according to an embodiment of this invention;

FIGS. 3a and 3b are graphical representations of programmed pulses according to an embodiment of this invention;

FIG. 4 is a block diagram of a programmed pulse delivery device according to an embodiment of this invention; and

FIG. 5 is a block diagram of a programmed pulse delivery device with a pneumatic actuated pump according to one embodiment of this invention.

### DETAILED DESCRIPTION OF THE INVENTION

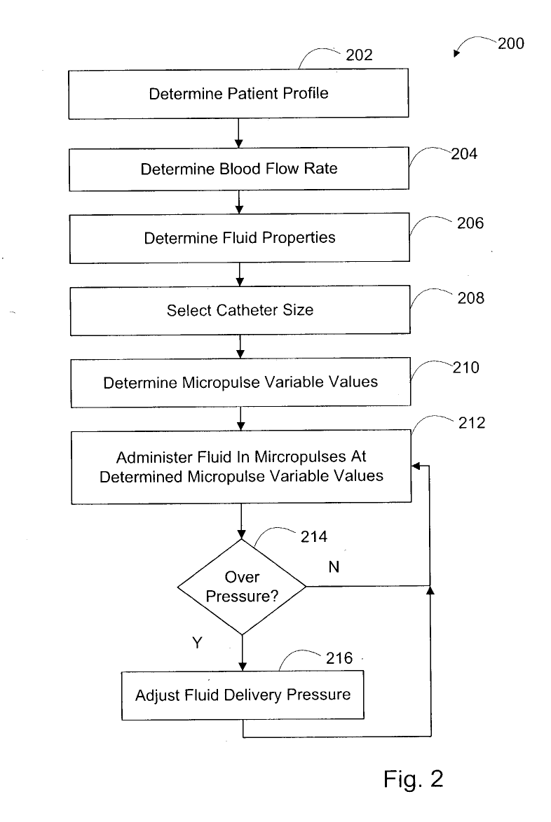

Referring to FIG. 2, a method for infusing or administering a fluid in programmed pulses, according to an embodiment of this invention, begins with determining a patient profile, step 202. A programmed pulse is generally a small volume of a fluid, such as a drug, in relatively short bursts, or otherwise at a low rate of infusion. A fluid denotes a substance in a form that takes the shape of its container, such as a substance in a liquid state. The patient profile generally refers to a characteristic or characteristics that may be useful in assessing a patient, such as weight, blood pressure, heart rate, temperature, etc. The profile is generally determined to assess the general condition of the patient, and to determine, in the instance the fluid is a drug, the dosage to be used for the infusion therapy. A drug generally denotes any substance that may be used in connection with a medical therapy, such as a pharmaceutical substance or composition, a medicine, a contrast agent used in X-Ray or magnetic resonance imaging (“MRI”), such as dyes, etc. A drug profile refers to the kinetic properties of drug delivery across the blood-brain border.

The patient profile may be determined with appropriate devices, such as a weight scale, blood pressure cuff, heart rate monitor, thermometer, etc., which are independent of the device for programmed pulse delivery of a fluid, e.g., the programmed pulse delivery device. In this instance, the patient profile or information related thereto, such as a dosage, may be noted for use in administering a fluid in programmed pulses as described herein, or input into the programmed pulse delivery device with an input device, such as a keypad or keyboard incorporated in or connected to the programmed pulse delivery device. Alternatively, the devices for determining the patients profile may interface with the programmed pulse delivery device, to provide patient profile data directly to the device.

In one embodiment, the blood flow rate of a blood vessel to be infused, e.g., the blood vessel that the fluid will be administered to, is determined, step 204. The blood flow rate may be determined in a variety of ways, such as with a blood flow sensor. The blood flow sensor may interface with the programmed pulse delivery device to provide the blood flow rate data directly to the programmed pulse delivery device. The blood flow sensor may be a stand-alone device independent of the programmed pulse delivery device. Blood flow data derived with the stand-alone sensor may similarly be noted for use in administering the fluid in programmed pulses or input into the programmed pulse delivery device with the input device. Alternatively, blood flow may be determined by estimating the blood flow based on the patient's profile. For example, blood flow data for a blood vessel, such as the carotid artery, for a plurality of patient profiles may be compiled and used to estimate or extrapolate the blood flow of the blood vessel for a particular patient based on the particular patient's profile. The compiled data for the plurality of patient profiles may be provided in a chart that may be used by medical professionals to manually estimate the blood flow or in database stored on a computer readable medium for estimating the blood flow with a programmed pulse delivery device.

Fluid properties relevant to streaming for the particular fluid to be infused may then be determined, step 206. Relevant fluid properties include static fluid properties, such as a fluid density, specific weight, specific gravity, viscosity, elasticity, etc., or kinetic properties. Where the fluid is a drug, the relevant fluid properties include pharmacological properties of the drug. Kinetic properties, generally relate to the transport or capability of being transported in the blood stream, and pharmacological properties relate to the effect or usefulness of the drug to be infused. For example, relevant kinetic properties may include streaming or anti-streaming properties, the typical volume rate of infusion, etc. Pharmacological properties may include, with respect to a particular fluid, data relating to the concentration of the drug in the blood vessel and the concentration of the blood during recirculation, protein binding, cerebral transit time, bio-phase equilibrium time, blood:brain partition coefficient, transfer rate across the blood:brain barrier, blood:brain transfer profile of the drug, active transport, drug formulation, rate of elimination or efflux from the brain, therapeutic index or concentration, maximum tolerable concentration, receptor efficacy, local metabolism in the brain, etc. The relevant fluid properties for fluids may be provided in chart form or in databases stored on a computer readable medium, which may be input or accessible to the programmed pulsed delivery device.

A catheter size for programmed pulsed infusion may then be selected, step 208. The catheter size is generally selected in accordance with the size of the blood vessel that is to be infused. The catheter size may also be input or accessible to the programmed pulsed delivery device.

At step 210, the value of a programmed pulse variable or variables are determined. Programmed pulse variables are variables that generally define a programmed pulse. Programmed pulse variables may include variables related to the timing of the programmed pulse, the volume of fluid infused, the pressure at which the fluid will be infused, etc. In one embodiment, programmed pulse variables that define a programmed pulse include a fluid flow rate, a frequency, and duration. A programmed pulse variable, in the instance the fluid is a drug, may include a drug concentration where for example the drug is included in a solution or suspension. Additionally, programmed pulse variables may include fluid delivery pressure.

The value or values of programmed pulse variables that define programmed pulses may be determined in a variety of ways. In one embodiment, the values are determined based at least partially on a fluid property relevant to streaming, such as a static fluid property, kinetic property, or where the fluid is a drug, based on a pharmacological property. In another embodiment, the values of programmed pulse variables are be based at least partially on the blood flow rate in the blood vessel to be infused. In yet another embodiment, the values are based at least partially on the patient profile. The values of programmed pulse variables are preferably determined by or in connection with the programmed pulse delivery device based on the patient profile, a fluid property relevant to streaming, or a blood flow rate in the blood vessel to be infused, or a combination thereof. For example, the programmed pulse delivery device may access fluid property data for the particular fluid to be administered and may determine the values of programmed pulse variables that will be used in defining programmed pulses for infusing the particular fluid. In one embodiment, a database including a set or sets of predefined values of programmed pulse variables, for a variety of patient profiles, blood flow rates, fluid properties, or catheter sizes, are stored on a computer readable medium. The programmed pulse delivery device may access the database to determine the appropriate values by looking up and/or computing the values based on the patient profile, blood flow rates, fluid properties relevant to streaming, or catheter size for a particular situation.

The fluid may then be administered in programmed pulses accordingly, step 212. The fluid may be administered in programmed pulses having programmed pulse variables with values determined or computed based on the patient profile, blood flow rate of the blood vessel to be infused, a fluid property relevant to streaming for the fluid to be infused, the catheter size to be used for infusing the fluid, or a combination thereof. In one embodiment, the fluid is administered into a blood vessel in programmed pulses having a flow rate of about 2% to 5% of the blood flow in the blood vessel to be infused and/or for a duration of 1-5 seconds. In one embodiment, the fluid is solution or suspension including a drug with a concentration that is determined based on the dosage and/or the drugs kinetic properties. Alternatively, the drug concentration may be determined based on a therapeutic index or concentration for the drug, or a blood:brain transfer profile. In one embodiment, the fluid is a drug and the duration is based on the transfer rate across the blood:brain barrier or blood:brain transfer profile or coefficient of the drug, receptor efficacy, or local metabolism in the brain. In another embodiment, the fluid is a drug and the frequency of the programmed pulses is determined based on the rate of elimination of efflux of the drug from the brain and/or the concentration of the drug if in a solution or suspension.

In one embodiment, the fluid delivery pressure is observed to prevent an overpressure condition, step 214. An overpressure condition generally refers to a condition in which the fluid delivery pressure exceeds a working pressure or pressure limit. A working pressure is generally a pressure less than the pressure limit, such as the pressure limit with an appropriate safety factor applied. For example, a working pressure for a pressure limit of 800 mm Hg with a Safety factor of 1.5 is 800 mm Hg./1.5=533 mm Hg. If at step 214 an overpressure condition is observed, the fluid delivery pressure may be adjusted, step 216, or the infusion may be stopped.

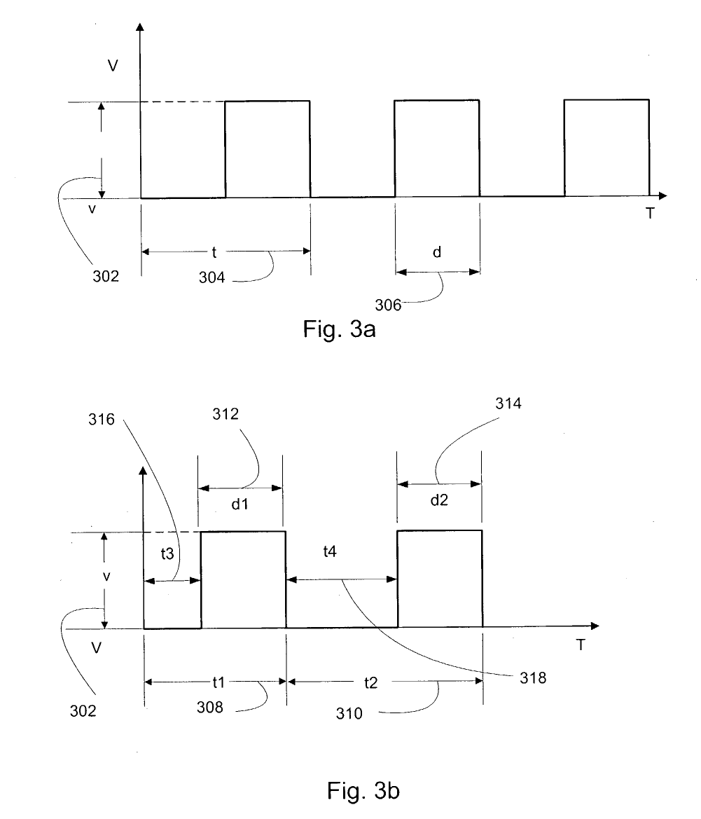

Referring to FIG. 3a, a graphical representation of a series of programmed pulses, according to one embodiment of this invention, is shown in terms of fluid flow (V) and time (T). A programmed pulse can generally be described as having a fluid flow rate (v) 302, a frequency (f), and duration (d) 306. The frequency may be described in terms of programmed pulses/time (t), e.g., 1.5 programmed pulses per second, etc. The amount of fluid administered per pulse is the flow rate (v) multiplied by the duration (d). The total amount of fluid administered is the amount of fluid administered per pulse multiplied by the frequency (f) and the total infusion time. In one embodiment, programmed pulses are administered at a fluid flow rate of about 5% of the blood flow in the blood vessel being infused and for a duration (d) of about 1 second until the desired dosage is achieved. Referring to FIG. 3b, the programmed pulses may be administered in a variable pattern. For example, the frequency (f) may vary for times (t1) 308 and (t2) 310. The duration as shown in with (d1) 312 and (d2) 314, the time frame between the pulses as shown with (t3) 316 and (t4) 318, and the flow rate (v) may vary over the infusion period. The fluid flow rate (v), frequency (f), and duration (d) programmed pulse variables may be determined or computed in either the fixed or variable programmed pulse embodiments based on the patient profile, blood flow rate, a fluid property relevant to streaming, catheter size, or a combination thereof.

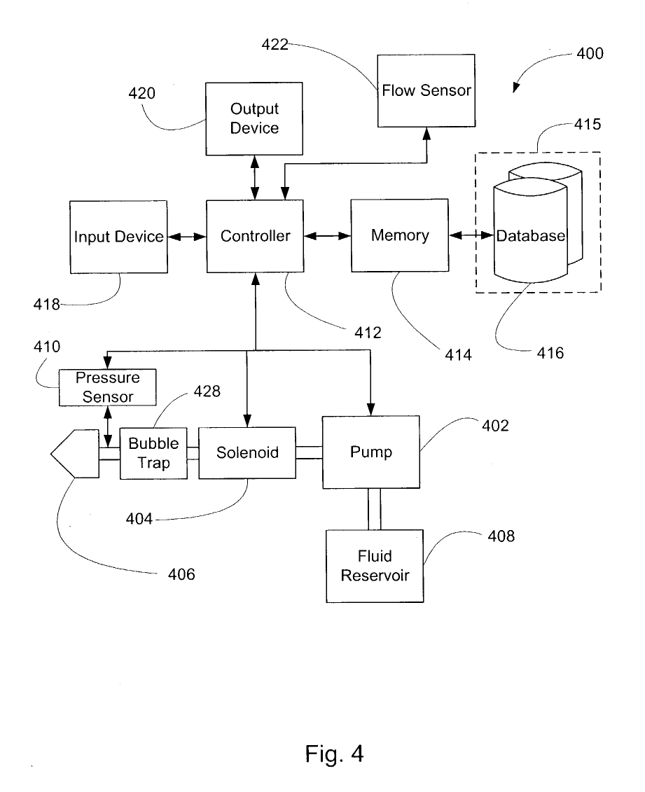

Referring to FIG. 4, a programmed pulse delivery device 400, in one embodiment, includes a pump 402, a controller 412 with associated computer memory 414, and a fluid reservoir 408. The controller generally provides a drive signal to drive the pump 402 for delivering the fluid in programmed pulses through an orifice, such as a catheter 406. The controller may be a micro-controller or processor that is capable of providing the drive signal to provide programmed pulse fluid infusion as described herein. The controller may be programmed or program code may be provided enabling the controller to access relevant data input by a user, such as the patient profile, blood flow rate, the particular fluid to be infused, fluid properties relevant to streaming, catheter size, values for programmed pulse variables, etc., for use in providing the drive signal. Alternatively, or in addition, the controller may access relevant data stored on at least one database, such as data of blood flow rates based on patient profiles, fluid properties relevant to streaming for particular fluids and concentrations, values for programmed pulse variables based on blood flow in blood vessel to be infused, fluid properties relevant to streaming, patient profiles, catheter size, or a combination therefore.

In one embodiment, the computer memory 414 has associated therewith at least one database 416. A database or databases may include data of blood flow for a blood vessel or vessels for a plurality of patient profiles, fluid property data relevant to streaming for particular fluids that may be infused, such as data of kinetic and pharmacological properties of a drug, values of programmed pulse variables based on a patient profile, blood flow rate, a fluid property relevant to streaming, and/or a catheter size. The databases may be stored on a computer readable medium that is accessed by the controller to provide the drive signal for programmed pulsed fluid infusion.

In one embodiment, the controller, computer memory, and a computer readable medium 415 are provided in a standalone computer, such as a personal computer, a special purpose computer, etc., that interfaces with the pump 402 to provide the drive signal for programmed pulsed infusion. The database or databases may be stored locally at the stand-alone computer, or remotely, such as on a server computer connected to the standalone compute over a communication network, such as a local area network (“LAN”), wide area network (“WAN”), etc. The databases may be stored on computer readable medium 415, such as a hard drive, optical media, magnetic tape, etc. Additionally, the computer readable medium 415 may include program code that when executed determines the values of programmed pulse variables based on a patient profile, blood flow in the blood vessel to be infused, fluid properties relevant to streaming for particular fluids, catheter size, or a combination thereof. In one embodiment, the stand-alone computer does not interface with the pump. In this instance, the values of programmed pulse variables may be determined and displayed to the user, which may then be input into a stand-alone pump capable of delivering a fluid in programmed pulsed fashion in accordance with the values of programmed pulse variables determined with the stand-alone computer.

In one embodiment, information, such as the patient profile, the particular fluid being administered, and the catheter size may be provided to the controller with an input device 418, such as a keypad, keyboard, mouse, touch pad, etc. The controller may display information with an output device 420. The output device may be a liquid crystal display (“LCD)”, a cathode ray tube (“CRT”) monitor, etc., which may also be a touch screen data input device. In one embodiment, the controller interfaces with a flow sensor 422, which provides the blood. flow rate in the blood vessel to be infused. A pressure sensor 410 interfacing with the controller may also be included for observing fluid delivery pressure.

The pump 402 may be any type of pumping apparatus, including, but not limited to a pneumatic actuated pump, etc. In one embodiment, the programmed pulse delivery device includes a control solenoid 404 that is actuated with a signal from the controller to deliver the fluid in programmed pulses as described herein. The device may also include a bubble trap 428 for removing air bubbles from the fluid being infused.

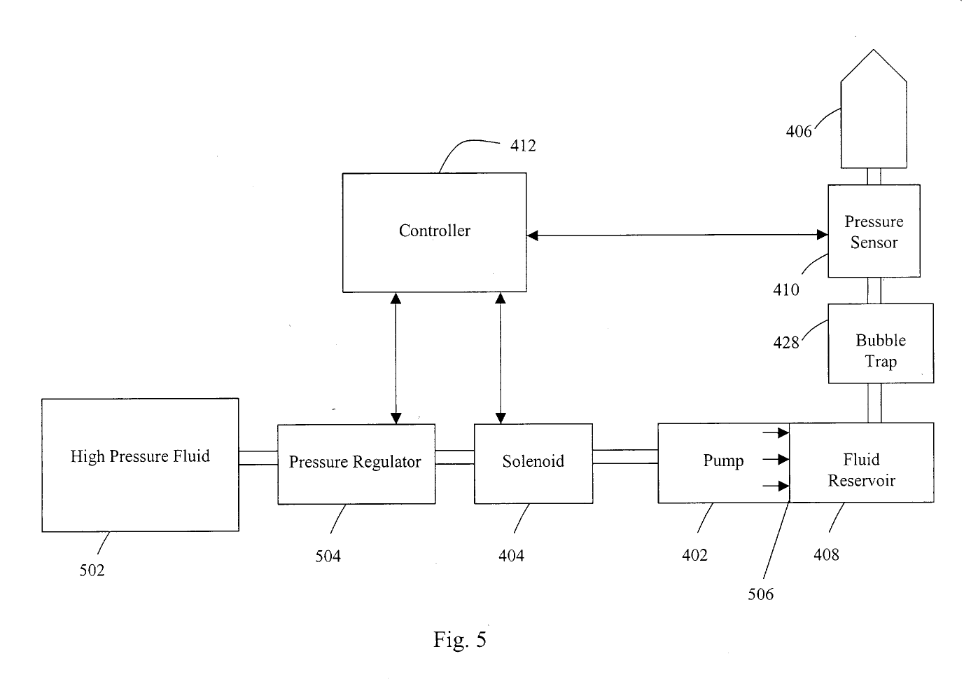

Referring to FIG. 5, a programmed pulse delivery device with a pneumatic actuated pump according to one embodiment of this invention includes a high-pressure actuating fluid source 502, which provides the actuating force for the pneumatic pump 402. The high-pressure actuating fluid source 502 may be any compressed gas, such as air, nitrogen, oxygen, etc. In one embodiment, the programmed pulse delivery device includes a pressure regulator 504 for regulating the high-pressure actuating fluid to a desired pressure, such as a working pressure or a pressure limit. The pressure regulator 504 may be set and adjusted manually or automatically by the controller 412. The control solenoid 404 receives the drive signal from the controller 412 to release the high-pressure or regulated actuating fluid to the pneumatic pump 402 for the pneumatic pump 402 to deliver the fluid in programmed pulses as described herein. In one embodiment, the pneumatic pump 402 includes a plunger 506 that extends with the application of the actuating force created by the high or regulated pressure actuating fluid. The plunger 506 extends an amount corresponding to the variables of the programmed pulses into or toward the fluid reservoir to expel the fluid from the fluid reservoir 408, such as a syringe. The expelled fluid delivered from the fluid in the reservoir in programmed pulses. In one embodiment, the syringe includes a Luer lock.

While the invention has been described and illustrated in connection with preferred embodiments, many variations and modifications, as will be evident to those skilled in the art, may be made without departing from the spirit and scope of the invention. The invention is thus not limited to the precise details of construction set forth above as such variations and modifications are intended to be included within the spirit and scope of the invention.

What is claimed is:

1. A method of infusing a fluid into a blood vessel comprising administering the fluid into the blood vessel in programmed pulses defined by programmed pulse variables comprising a fluid flow rate, a frequency, and a duration, values of the programmed pulse variables determined based at least in part on at least one of a fluid property of the fluid to be infused relevant to streaming, blood flow in the blood vessel to be infused, a catheter size, and a patient profile.

2. The method of claim 1, wherein the fluid is administered in programmed pulses independent of diastole.

3. The method of claim 1, comprising administering the fluid in programmed pulses into a carotid artery.

4. The method of claim 3, comprising administering the fluid at a fluid flow rate of about 2% to about 5% of arterial blood flow of the carotid artery.

5. The method of claim 1, wherein the fluid property of the fluid to be infused relevant to streaming comprises one of a static fluid property and a kinetic fluid property.

6. The method of claim 1, wherein the fluid comprises a drug and the fluid property of the fluid to be infused relevant to streaming comprises a pharmacological property of the drug.

7. The method of claim 1, comprising administering the fluid in programmed pulses at the duration of about one to five seconds.

8. The method of clam 1, wherein the fluid comprises a drug and wherein the drug is administered in programmed pulses at the duration based on a transfer rate of the drug across a blood brain barrier.

9. The method of clam 1, wherein the fluid is a drug and wherein the drug is administered in programmed pulses at the frequency based on a rate of elimination of the drug from a brain.

10. The method of claim 1, wherein the fluid comprises a drug, the drug administered in programmed pulses in a carotid artery to treat a condition selected from the group consisting of stroke after thrombosis, cancer, cerebral vasospasm, infection, and localization of brain function.

11. The method of claim 1, wherein the fluid comprises a drug having a drug concentration determined based on a kinetic property of the drug.

12. The method of claim 1, wherein the fluid comprises a drug having a drug concentration determined based on a therapeutic index of the drug.

13. A programmed pulse infusion device comprising:
a pump;
a controller with associated memory interfacing with the pump; and
a fluid reservoir containing a fluid feeding the pump, the controller providing a drive signal for the pump to deliver the fluid for infusion into a blood vessel in programmed pulses defined by programmed pulse variables comprising a fluid flow rate, a frequency, and a duration, values of the programmed pulse variables determined based at least in part on at least one of a fluid property of the fluid to be infused relevant to streaming, blood flow in the blood vessel to be infused, a catheter size, and a patient profile.

14. The programmed pulse infusion device of claim 13, wherein the fluid is delivered in programmed pulses independent of diastole.

15. The programmed pulse infusion device of claim 13, wherein the fluid is delivered for infusion into a carotid artery.

16. The programmed pulse infusion device of claim 13, wherein the fluid property of the fluid to be infused relevant to streaming comprises one of a static fluid property and a kinetic fluid property.

17. The programmed pulse infusion device of claim 13, wherein the fluid comprises a drug and the fluid property of the fluid to be infused relevant to streaming comprises a pharmacological property of the drug.

18. The programmed pulse infusion device of claim 13, wherein the fluid comprises a drug infused in programmed pulses at the duration based on a transfer rate of the drug across a blood brain barrier.

19. The programmed pulse infusion device of claim 13, wherein the fluid comprises a drug infused in programmed pulses at the frequency based on a rate of elimination of the drug from a brain.

20. The programmed pulse infusion device of claim 13, comprising a control solenoid interfacing with the controller providing an actuating signal to the solenoid to deliver the fluid in programmed pulses and wherein the pump comprises a pneumatic actuated pump.

21. The programmed pulse infusion device of claim 20, wherein the fluid reservoir comprises a syringe and the pneumatic actuated pump comprises a plunger extended by an actuating force toward the syringe to deliver the fluid from the syringe in programmed pulses.

22. The programmed pulse infusion device of claim 13, comprising a bubble trap removing air bubbles from the fluid being delivered.

23. The programmed pulse infusion device of claim 13, comprising a pressure sensor interfacing with the controller.

24. A computer readable medium comprising program code that when executed enables a user to determine values of programmed pulse variables for infusing a fluid in a blood vessel in programmed pulses defined by the programmed pulse variables comprising a fluid flow rate, a frequency, and a duration, the values of programmed pulse variables determined at least in part on at least one of a fluid property of the fluid to be infused relevant to streaming, blood flow in the blood vessel to be infused, a catheter size, and a patient profile.

25. The computer readable medium of claim 24, wherein the values of the programmed pulse variables are determined independent of diastole.

26. The computer readable medium of claim 24, wherein the program code enables users to determine values of programmed pulses variables for infusing the fluid into a carotid artery.

27. The computer readable medium of claim 24, wherein the fluid property of the fluid to be infused relevant to streaming comprises one of a static fluid property and a kinetic fluid property.

28. The computer readable medium of claim 24, wherein the fluid comprises a drug and the fluid property of the fluid to be infused relevant to streaming comprises a pharmacological property of the drug.

29. The computer readable medium of claim 24, wherein the fluid comprises a drug infused in programmed pulses in a carotid artery to treat a condition selected from the group consisting of stroke after thrombosis, cancer, cerebral vasospasm, infection, and localization of brain function.

30. A computer system comprising a controller and associated computer memory, and a computer readable medium accessible to the controller, stored on the computer readable medium at least one database comprising at least one of:
data of fluid properties for a fluid that may be infused into a blood vessel relevant to streaming; and
values of programmed pulse variables based on at least one of a patient profile, a blood flow rate in the blood vessel , a fluid property for the fluid relevant to streaming, and a catheter size, the database accessed to enable users to determine the values of programmed pulse variables for infusing the fluid in the blood vessel in programmed pulses defined by the programmed pulse variables comprising a fluid flow rate, a frequency, and a duration.

31. The computer system of claim 30, wherein the values of programmed pulse variables based on a fluid property for the fluid relevant to streaming comprises at least one of a static fluid property and a kinetic fluid property.

32. The computer system of claim 30, wherein the fluid comprises a drug and wherein the values of programmed pulse variables based on a fluid property for the fluid relevant to streaming comprises a pharmacological property of the drug.

33. The computer system of claim 30, wherein the fluid comprises a drug and wherein the duration of a programmed pulse is based on a transfer rate of the drug across a blood brain barrier.

34. The computer system of claim 30, wherein the fluid comprises a drug and wherein the frequency of a programmed pulse is based on a rate of elimination of the drug from a brain.
